(19) United States
(12) Patent Application Publication
(10) Pub. No.: US 2007/0010754 A1
(43) Pub. Date: Jan. 11, 2007

(54) Method for initiating occupant-assisted measures inside a vehicle

(76) Inventors: Klaus-Robert Muller, Berlin (DE); Benjamin Blankertz, Berlin (DE); Gabriel Curio, Berlin (DE)

Correspondence Address: BIRCH STEWART KOLASCH & BIRCH, PO BOX 747, FALLS CHURCH, VA, 22040-0747, US

(21) Appl. No.: 10/549,701

(22) PCT Filed: Mar. 22, 2004
(86) PCT No.: PCT/EP04/03012

§ 371 Date: Aug. 3, 2006

(30) Foreign Application Priority Data
Mar. 20, 2003 (DE) 103 12 519.1

(51) Int. Cl. A61B 5/04; B60K 28/00

(52) U.S. Cl. 600/544; 180/272

(57) ABSTRACT

In the method for initiating occupant-assisted measures inside a vehicle, particularly a motor vehicle, cerebral-current signals of at least one vehicle occupant, particularly of the driver, are detected by a measurement technique. On the basis of the cerebral-current signals, the intention of the vehicle occupant is estimated or detected by real-time processing. Based the intention of the vehicle occupant, measures for transferring the current state of the vehicle into a state of the vehicle matched to the intention of the vehicle occupant are initiated in advance.

The invention relates to a method for initiating occupant-assisted measures inside a vehicle.

From DE 198 01 009 C1, a method is known wherein an emergency or stress situation of the driver of a vehicle is detected and a device for initiation or performing a braking process is actuated for support. In doing so, the emergency or stress situation of the driver is detected with the aid of sensors provided to detect a change of the blood pressure and/or a change of the pulse and/or a change of the pupil and/or a change of the facial expression and/or a change of the eyelid reflex and/or a muscular contraction, preferably a muscular contraction of the hand, and/or a change of the skin resistance and/or a change of the sweat secretion.

The time duration up to the generation of one of the above mentioned physical reactions on an emergency or stress situation perceived by the driver will cause a delay in the supportive initiation of the braking process, which may be disadvantageous.

Further, from DE 197 02 748 A1, it is known to detect the condition of the conductor of a vehicle, e.g. of a train, by monitoring, for instance, the cerebral currents of the conductor.

It is an object of the invention to provide a method for initiating occupant-assisted measures inside a vehicle wherein the time span between the generation of the intention e.g. of the driver of the vehicle and the to-be-initiated measure is abbreviated and the measure can thus be initiated virtually without time delay.

According to the invention, to achieve the above object, there is proposed a method for initiating occupant-assisted measures inside a vehicle wherein
cerebral-current signals of at least one vehicle occupant, particularly of the driver, are detected by a measurement technique,
on the basis of the cerebral-current signals, the intention of the vehicle occupant is estimated or detected by real-time processing, and
on the basis of the intention of the vehicle occupant, measures for transferring the current state of the vehicle into a state of the vehicle matched to the intention of the vehicle occupant are initiated in advance.

Advantageous embodiments of the invention are indicated in the subclaims.

According to the invention, the action-specific intentions of the occupants and the driver, respectively, are detected on the basis of their cerebral currents. This is performed at the earliest possible point of time so that delays which might occur e.g. up to the generation of secondary reactions of the body, will be avoided. Further, also intentions which do not cause secondary reactions of the body can be detected. For instance, on the basis of the cerebral currents, it can be detected in what manner the driver intends to steer the vehicle, thus allowing for optimum preparation of vehicle stabilization systems in accordance with the type of the steering maneuver.

Thus, according to the invention, there is proposed a method for use in vehicles in order to provide an improved driver/vehicle interface by evaluation of cerebral currents, e.g. by EEG, MEG, NIRS, fMRI and/or EMG.

The method according to the invention has the property, inter alia, that the driver's attitude in a very general sense and, especially, the driver's reaction errors and reaction delays are detected and analyzed and thus, as a novel multi-purpose feature for improved vehicle safety, will be available to be inputted into a safety system arranged downstream. The method can be used in a vehicle, inter alia, for the purposes of

1. accident-preventive safety measures such as
a) automatic safety belt tightening
b) seat optimization
c) optimization of the vehicle reagibility to prepare a braking/steering operation
d) pre-optimization of the vehicle dynamics in case of time-critical decisions
e) all predicative safety measures.

2. driver-based verification of device-detected hazardous situations such as, e.g.
a) detection of a congruent motor generation of an intention
b) situation modeling and validating.

3. continuous vigilance monitoring.

The invention, its foundations and principal ideas will be described in greater detail hereunder.

The invention allows for a basically novel quality of man/machine interfaces by the combination of cerebro-physiological findings and algorithmic developments in the field of information technology, notably in that the concept of a direct transformation of cerebral signals into machine-related control commands is realized in a brain/computer interface (BCI) as a real-time implementation. As a non-invasive measurement method which in principle is suited for everyday applications, use is made e.g. of the multi-channel EEG with a time resolution in the milliseconds range. The methodological approach is based on robust algorithms of machine learning and signal processing for extraction, identification and classification of EEG cerebral signals which represent intentions of natural motions in psychophysiologically well-defined interaction situations between humans and the environment. A further characteristic feature of the BBCI used here resides in the adaptation to a training situation optimized for the user; in this training situation, in contrast to other BCI methods, the user does not need to undergo several training sessions but merely one about 20-minute-long training phase to thus obtain starting material for the learning algorithm (cf. Blankertz, B., Curio, G., Müller, K.-R. (2003), Classifying Single Trial EEG: Towards Brain Computer Interfacing, Advances in Neural Information Processing Systems 14, eds. T. G. Dietterich, S. Becker and Z. Ghahramani, MIT Press: Cambridge, Mass., 157-164; Dornhege, G., Blankertz, B., Curio, G., Müller, K.-R., Combining Features for BCI, Advances in Neural Information Processing Systems 15, eds. S. Becker, S. Thrun and K. Obermayer, MIT Press: Cambridge, Mass. (2003)).

For a BCI, well-defined application perspectives for clinical use in paralyzed patients do already exist on an international level, particularly for cases of complete paraplegia. The invention for the first time opens up the possibility, in time-critical real-time applications as typically existing e.g. in driver/vehicle interfaces, to realize novel methodical approaches:
1. In the psychophysiological research for detection and handling of reaction errors and reaction delays of the driver, it is now for the first time possible, both in virtual driving simulations and in real driving situations, to detect the motor reaction intentions of the driver with high time resolution in the millisecond range as non-averaged individual results and thereby analyze them in dependence on the currently varying perceptual context (multi-modal environment information as well as instrument signals).
2. When used as a driver assistance system, concepts of “integrated safety” can be enriched by novel components for a continuously proceeding (“on-the-fly”) driver modeling:
   a) Due to the BBCI real-time suitability, the EEG correlatives—identifiable as individual events—of intention generation and specific motion preparations can serve as a novel input value for concepts of accident-preventive safety, e.g., in automobiles, for the purposes of motor-powered safety belt tightening, seat optimization or optimization of the vehicle reagibility in order to prepare a braking/steering operation.
   b) Moreover, a quickest possible driver-based “verification” of the realization of hazards can be performed in a machine-operated (e.g. optical) manner by detection of a congruent motor intention buildup of the driver, allowing for a correspondingly validated situation modeling.
   c) Particularly, time-critical decision alternatives such as e.g. a choice, dictated by the situation, between an emergency braking maneuver and a well-steered dodging maneuver which are legally left to the driver's discretion, can be prognosticated already tenths of seconds before the actual reaction motion of the driver by extracting the corresponding motor intentions from the EEG signal of the driver and utilizing them for the purposes of a pre-optimization of the vehicle dynamics.

As an additive advantage offered by this EEG-based BCI approach, mention should be made of the farther-reaching multi-purpose feature that these EEG data, apart from the novel applications defined here, also allow for a seamless integration of concepts for continuous driver vigilance monitoring which were established already in the past.

1. A method for initiating occupant-assisted measures inside a vehicle, particularly a motor vehicle, wherein
cerebral-current signals of at least one vehicle occupant, particularly of the driver, are detected by a measurement technique,
on the basis of the cerebral-current signals, the intention of the vehicle occupant is estimated or detected by real-time processing, and
on the basis of the intention of the vehicle occupant, measures for transferring the current state of the vehicle into a state of the vehicle matched to the intention of the vehicle occupant are initiated in advance.

2. The method according to claim 1, characterized in that the physiological signals are detected non-invasively.

3. The method according to claim 1 or 2, characterized in that the cerebral-current signals are cerebral signals such as e.g. EEG, MEG, NIRS, fMRI and/or EMG.

4. The method according to claim 1, characterized in that the real-time processing of the measurement signals is performed by use of methods of signal processing and/or machine learning which allow an evaluation of the measurement signals as individual signals and without extensive training of the occupant of the vehicle.

5. The method according to claim 4, characterized in that the methods for signal processing for adaptive feature extraction from the measurement signals comprise, alternatively or in any desired combination, at least one of the following features:
a) filtration (spatial and in the frequency range) and downsampling,
b) splitting and projection, respectively,
c) determination of spatial, temporal or spatio-temporal complexity dimensions,
d) determination of coherence dimensions (related to phase or band energy) between input signals.

6. The method according to claim 5, characterized in that the filtration comprises, alternatively or in any desired combination, at least one of the following features:
a) wavelet or Fourier filter (short-time),
b) FIR or IIR filter,
c) Laplace and common average reference filter,
d) smoothing method.

7. The method according to claim 5, characterized in that the splitting and projection, respectively, comprises, alternatively or in any desired combination, at least one of the following features:
a) independent component analysis and main component analysis,
b) projection pursuit technique,
c) sparse decomposition techniques,
d) common spatial patterns techniques,
e) common substance decomposition techniques,
f) (Bayes') sub-space regularization techniques.

8. The method according to claim 4 or any one of the preceding claims as far as dependent on claim 4, characterized in that the machine learning method comprises a classification and/or regression, notably by use of
a) core-based linear and non-linear learning machines (e.g. support vector machines, Kern Fisher, linear programming machines),
b) discriminance analyses,
c) neuronal networks,
d) decision trees,
e) generally, all linear and non-linear classification methods for the features obtained by signal processing.

9. The method according to claim 1, characterized in that the initiating measures are accident-preventive measures such as e.g.
a) automatic safety belt tightening,
b) seat optimization,
c) optimization of the vehicle reagibility to prepare a braking/steering operation,
d) stability computations,
e) pre-optimization of the vehicle dynamics in case of time-critical decisions,
f) all predicative safety measures.

10. The method according to claim 1, characterized in that the intention or estimated on the basis of the cerebral-current signals serves for the verification of device-detected hazard situations, particularly by detection of a congruent motor intention build-up and situation modeling and validating.

11. The method according to claim 1, characterized by use and integration continuous vigilance monitoring.

12. The method according to claim 1, characterized in that the measures to be initiated are taken on the basis of an averaging of the intentions of a plurality of vehicle occupants.
